SEQUENCE LISTING (1 sequence)

SEQ ID NO: 1 - protein, 91 aa, Homo sapiens
  ref <308>: DATABASE ACCESSION NUMBER: Swiss-Prot entry P80511
  ref <309>: DATABASE ENTRY DATE: 1996-01-10
  ref <313>: RELEVANT RESIDUES IN SEQ ID NO: (1)..(91)
TKLEEHLEGIVNIFHQYSVRKGHFDTLSKGELKQLLTKELANTIKNIKDKAVIDEIFQGLDANQDEQVDFQEFISLVAIALKAAHYHTHKE

(19) United States
(12) Patent Application Publication
(10) Pub. No.: US 2011/0312922 A1
(43) Pub. Date: Dec. 22, 2011

(54) EDTA Resistant S100A12 Complexes (ERAC)

(75) Inventors: Magne K. Fagerhol, Oslo (NO); Annette Larsen, Tolvsrod (NO); Tor Magne Madland, Nesttun (NO); Johan G. Brun, Paradis (NO)

(73) Assignee: ERAC AS, Tolvsrod (NO)

(21) Appl. No.: 12/738,579

(22) PCT Filed: Oct. 17, 2008
(86) PCT No.: PCT/EP08/64058

§ 371 Date: Sep. 3, 2010

Related U.S. Application Data
(60) Provisional application No. 60/999,653, filed on Oct. 19, 2007.

(30) Foreign Application Priority Data
Oct. 19, 2007 (DK) PA 2007 01511

(51) Int. Cl. A61K 31/616; G01N 33/566; C07K 14/47; A61P 31/00; A61P 37/06; A61P 35/00; A61P 13/12; A61P 9/00; C12N 5/071; A61P 29/00

(52) U.S. Cl. 514/165; 435/325; 435/7.92; 436/501; 435/7.21; 530/409

(57) ABSTRACT

The present invention relates to the discovery of a new protein complex in human biological materials that can be used for diagnostic and other purposes. The invention further relates to products, methods and uses related to said protein complex.

This application is a non-provisional of U.S. provisional application Ser. No. 60/999,653 filed 19 Oct. 2007, which is hereby incorporated by reference in its entirety.

All patent and non-patent references cited in the application, or in the present application, are also hereby incorporated by reference in their entirety.

### FIELD OF INVENTION

The present invention relates to the discovery of a new protein complex in human biological materials that can be used for diagnostic and other purposes.

### BACKGROUND OF INVENTION

Human white blood cells contain many different proteins that are important for their biological functions, for instance killing and removal of microorganisms, tumor cells or foreign bodies; degradation and removal of dead tissue; recruitment of more leukocytes to sites of pathological processes; up- or down regulation of inflammation. During activation of leukocytes, for instance neutrophil granulocytes or monocytes, they will phagocytose (engulf) foreign cells or materials as well as release effector proteins to the tissue and body fluids. By consequence, increased concentrations of leukocyte derived proteins will be found in body fluids or excretions like blood, cerebrospinal fluid, synovial fluid, crevicular fluid, nasal and bronchial secretions, saliva, urine or stools. Assays of substances from leukocytes in such materials are part of routine diagnostic procedures in many different patient groups. In addition to tell that pathology is indeed present, elevated levels of some markers may be specific for certain diseases and the type of pathological process.

Proteins belonging to the S100 family of proteins have contributed to this particular field. In particular, calprotectin, which is a heterotrimer of S100A8 and S100A9 has been widely used (Johne B & al. Mol. Pathol. 1997 June; 50(3):113-23). In recent years, interesting findings have also been reported for S100A12 (in the following abbreviated as A12). For instance, increased concentration of this protein can be found in serum from patients with inflammatory bowel disease (Foell D & al., Gut 2003; 52:847-853) and rheumatoid arthritis (Foell D & al., Arthritis & Rheumatism 2004; 50:1286-1295)) and in stool samples from patients with inflammatory bowel disease (Kaiser T & al., 2007, Gut. 2007 Aug. 3).

### SUMMARY OF INVENTION

In one aspect of the present invention there is provided a kit for detecting the presence of ERAC in a sample wherein divalent metal ions have been removed, said kit comprising

i) a solid support,
ii) a first targeting species bound to the solid support, said targeting species being capable of directly detecting ERAC when it is present in a sample that is brought into contact with the solid support, and
iii) at least one label.

In an embodiment of the invention, the kit further comprises a carrier molecule, preferably a polymeric carrier molecule. A polymeric carrier molecule according to the invention preferably comprises reactive, functional groups in an amount of from about 5 to about 5,000 micro moles per gram of polymeric carrier.

In another embodiment the polymeric carrier molecule comprising e.g. a dextran chain according to the invention comprises less than about 400 labelling species, preferably in the form of visibly detectable targeting species or fluorescently detectable labelling species, such as less than 380 labelling species, for example less than 360 labelling species, such as less than 340 labelling species, for example less than 320 labelling species, such as less than 300 labelling species, for example less than 280 labelling species, such as less than 260 labelling species, for example less than 240 labelling species, such as less than 220 labelling species, for example less than 200 labelling species, such as less than 180 labelling species, for example less than 160 labelling species, such as less than 140 labelling species, for example less than 120 labelling species, such as less than 100 labelling species, for example less than 80 labelling species, such as less than 70 labelling species, for example less than 60 labelling species, such as less than 50 labelling species, for example less than 40 labelling species, such as less than 30 labelling species, for example less than 25 labelling species, such as less than 20 labelling species, for example less than 15 labelling species, such as less than 12 labelling species, for example less than 10 labelling species, such as less than 8 labelling species, for example less than 4 labelling species, such as less than 3 labelling species, for example less than 2 labelling species.

The molecular weight of a polymeric dextran chain can be about 500,000 Da, but molecular weights of from about 100,000 Da to about 900,000 Da can also be used.

Each dextran chain in one embodiment comprises approximately 2,700 glucose units of which 20-22% are activated, preferably with divinyl sulfon, although other connecting moieties can also be used as described herein below in detail.

Suitable polymeric carriers can be, for example, polymeric carriers with functional groups such as:

O— (e.g. deprotonated phenolic hydroxy groups, such as deprotonated aromatic hydroxy groups in tyrosine residues of polypeptides or proteins),
S— (e.g. deprotonated thiol groups on aromatic rings or aliphatic groups, such as deprotonated thiol groups in cysteine residues of polypeptides or proteins),
OH (e.g. aliphatic hydroxy groups on sugar rings, such as glucose or other monosaccharide rings in oligo- or polysaccharides; or alcoholic hydroxy groups in polyols, such as polyethylene glycols; or hydroxy groups in certain amino acid residues of polypeptides or proteins, such as serine or threonine residues),
SH (e.g. thiol groups in cysteine residues of polypeptides or proteins), primary amino groups (e.g. in lysine or ornithine residues of polypeptides or proteins; or in amino-substituted sugar rings in certain polysaccharides or derivatives thereof, such as chitosan) or secondary amino groups (e.g. in histidine residues of polypeptides or proteins).

Accordingly, the functional group in question on molecular species in the context of the invention will also normally be a nucleophilic function, such as a nucleophilic function of one of the above-described types.

In one embodiment about half of the about 600 connecting moieties, preferably, but not limited to, divinyl sulfon groups, per dextran chain react with targeting species, such as antibody, such as preferably an anti-ERAC monoclonal antibody, and labelling species, such as a label as described herein below, according to the invention, and this provides the figure of less than about 400 labelling species per dextran chain. However, it is clear that more than about half of the 600 connecting moieties may well react with a labelling species, and the number of labelling species may therefore be higher than about 400.

The number of labelling species and targeting species in a single polymeric carrier molecule according to the invention influences the minimum amount ERAC that can be detected according to the invention.

In one embodiment the minimum amount of ERAC detectable by a kit or method according to the invention is less than 1000 nanograms per millilitre sample, such as less than 900 nanograms per millilitre, such as less than 800 nanograms per millilitre, such as less than 700 nanograms per millilitre, such as less than 600 nanograms per millilitre, such as less than 500 nanograms per millilitre, such as less than 400 nanograms per millilitre, such as less than 300 nanograms per millilitre, such as less than 200 nanograms per millilitre, such as less than 100 nanograms per millilitre, such as less than 95 nanograms per millilitre, for example less than 90 nanograms per millilitre, such as less than 85 nanograms per millilitre, for example less than 70 nanograms per millilitre, such as less than 75 nanograms per millilitre, for example less than 70 nanograms per millilitre, such as less than 65 nanograms per millilitre, for example less than 60 nanograms per millilitre, such as less than 55 nanograms per millilitre, for example less than 50 nanograms per millilitre, such as less than 45 nanograms per millilitre, for example less than 40 nanograms per millilitre, such as less than 35 nanograms per millilitre, for example less than 30 nanograms per millilitre, such as less than 25 nanograms per millilitre, for example less than 20 nanograms per millilitre, such as less than 15 nanograms per millilitre, for example less than 10 nanograms per millilitre, such as less than 5 nanograms per millilitre, for example less than 1 nanograms per millilitre, such as less than 0.5 nanograms per millilitre, for example less than 0.1 nanograms per millilitre, such as less than 0.05 nanograms per millilitre, for example less than 0.01 nanograms per millilitre, such as less than 0.005 nanograms per millilitre, for example less than 0.001 nanograms per millilitre sample.

In another aspect there is provided a method of manufacturing a kit according to the invention, said method comprising the steps of

i) providing a solid support,
ii) adding to a binding zone of said solid support a labelled first targeting species capable of directly detecting ERAC when it is present in a sample that is brought into contact with the solid support, and
iii) adding to a test zone of said solid support a non-labelled first targeting species.

In yet another aspect there is provided a use of a kit according to the invention for the detection or quantification of ERAC in a sample.

In a further aspect there is provided a method for detection of ERAC comprising the steps of

i) providing a sample,
ii) removing divalent metal ions from said sample, thereby reducing or eliminating the amount of free divalent metal ion in the sample,
iii) providing a labelled first targeting species capable of directly detecting ERAC when present in said sample,
iv) bringing said treated sample into contact with said labelled first targeting species, and
v) detecting the presence of ERAC bound to said labelled first targeting species.

In an embodiment of the present invention, the method for detection of ERAC yields either a positive or a negative result. A lower cut-off level for the detection of ERAC may be pre-determined in order to set a lower limit for what constitutes a positive result of the method.

In one embodiment of the present invention, the ERAC detected has a molecular weight substantially identical to a molecular weight of native S100A12 oligomers comprising the same number of monomer units. In another embodiment, the detected ERAC has an aberrant molecular weight when compared to native S100A12 oligomers comprising the same number of monomer units. The weight of the ERAC detected by a kit or method according to the present invention may for instance be less than 50% as compared to native S100A12 oligomers comprising the same number of monomer units, or less than 60% compared to native S100A12 oligomers comprising the same number of monomer units, or less than 70% compared to native S100A12 oligomers comprising the same number of monomer units, or less than 80% compared to native S100A12 oligomers comprising the same number of monomer units, or less than 90% compared to native S100A12 oligomers comprising the same number of monomer units, or at least 110% compared to native S100A12 oligomers comprising the same number of monomer units, or at least 120% compared to native S100A12 oligomers comprising the same number of monomer units, or at least 130% compared to native S100A12 oligomers comprising the same number of monomer units, or at least 140% compared to native S100A12 oligomers comprising the same number of monomer units, or at least 150% compared to native S100A12 oligomers comprising the same number of monomer units, or at least 200% compared to native S100A12 oligomers comprising the same number of monomer units, or at least 250% compared to native S100A12 oligomers comprising the same number of monomer units, or at least 500% compared to native S100A12 oligomers comprising the same number of monomer units.

The ERAC detected with a kit or method according to the present invention may be in the form of a dimmer comprising two monomeric units, a trimer comprising three monomeric units, a tetramer oligomer comprising 4 monomeric units, a pentamer oligomer comprising 5 monomeric units, a hexamer oligomer comprising 6 monomeric units, a heptamer oligomer comprising 7 monomeric units, an octamer oligomer comprising 8 monomeric units, a nonamer oligomer comprising 9 monomeric units, or a decamer oligomer comprising 10 monomeric units. The ERAC detected may comprise a number of monomeric units such as 1, 2, 3, 4, 5, 6, 7, 8, 9, 10, 11, 12, 13, 14, 15, 16, 17, 18, 19 or 20 or a number of monomeric units above 20.

The molecular weight of the ERAC detected with a kit or method according to the present invention may be in the range of 500-2100 kDa, such as 500-1000 kDa or 750-1500 kDa, or 1000-2100 kDa, for instance in the range of 500-700 kDa, such as in the range of 550-600 kDa, such as in the range of 600-650 kDa, such as in the range of 650-700 kDa, or for instance in the range of 700-900 kDa, such as in the range of 700-750 kDa, such as in the range of 750-800 kDa, such as in the range of 800-850 kDa, such as in the range of 850-900 kDa, or for instance in the range of 900-1100 kDa, such as in the range of 900-950 kDa, such as in the range of 950-1000 kDa, such as in the range of 1000-1050 kDa, such as in the range of 1050-1100 kDa, or for instance in the range of 1100-1300 kDa, such as in the range of 1100-1150 kDa, such as in the range of 1150-1200 kDa, such as in the range of 1200-1250 kDa, such as in the range of 1250-1300 kDa, or for instance in the range of 1300-1500 kDa, such as in the range of 1300-1350 kDa, such as in the range of 1350-1400 kDa, such as in the range of 1400-1450 kDa, such as in the range of 1450-1500 kDa, or for instance in the range of 1500-1700 kDa, such as in the range of 1500-1550 kDa, such as in the range of 1550-1600 kDa, such as in the range of 1600-1650 kDa, such as in the range of 1650-1700 kDa, or for instance in the range of 1700-1900 kDa, such as in the range of 1700-1750 kDa, such as in the range of 1750-1800 kDa, such as in the range of 1800-1850 kDa, such as in the range of 1850-1900 kDa, or for instance in the range of 1900-2100 kDa, such as in the range of 1900-1950 kDa, such as in the range of 1950-2000 kDa, such as in the range of 2000-2050 kDa, such as in the range of 2050-2100 kDa.

In yet a further aspect there is provided a method for quantifying the amount of ERAC present in a sample, said method comprising the steps of

i) providing a kit according to the invention,
ii) providing a sample,
iii) bringing said sample into contact with said kit,
iv) detecting the presence of ERAC in said sample, and
v) quantifying the amount of ERAC detected in said sample.

In still a further aspect there is provided a method for quantifying the amount of ERAC present in a sample, said method comprising the steps of

i) performing a method according to the invention for the detection of ERAC, and
ii) quantifying the amount of ERAC bound to said labelled first targeting species.

In yet a further aspect there is provided a method for profiling of a sample or an individual, said method comprising the steps of

i) detecting the presence in a sample of ERAC according to the invention, or quantifying in a sample the amount of ERAC according to the invention, and
ii) qualitatively or quantitatively detecting the presence in said sample of at least one other immunological marker.

Preferably said other immunological marker is a member of the S100 family of proteins. More preferably said other immunological marker is selected from the group consisting of the proteins S100A1, S100A2, S100A3, S100A4, S100A5, S100A6, S100A7, S100A8, S100A9, S100A10, S100A11, S100A13, S100A14, S100A15, and S100A16.

Preferably said other immunological marker is calprotectin.

The members of the S100 protein family have been further described in e.g. Heizmann C W, Ackermann G E, Galichet A: “Pathologies involving the S100 proteins and RAGE”, Subcell Biochem. 2007; 45:93-138 and Marenholz I, Heizmann C W, Fritz G: “S100 proteins in mouse and man: from evolution to function and pathology (including an update of the nomenclature)”, Biochem Biophys Res Commun, 2004 Oct. 1; 322(4):1111-22.

In still a further aspect there is provided a method for monitoring the presence of ERAC in sample, said method comprising the steps of

i) detecting the presence in a body fluid sample of ERAC according to the invention, or quantifying in a sample the amount of ERAC according to the invention, and
ii) repeating step (i), optionally at predetermined intervals.

The monitoring of ERAC is preferably performed at regular intervals, such as intervals of seconds, minutes, hours, days, weeks or months, or years. Preferably the monitoring is performed every year, such as 2 times a year, such as 3 times a year, such as 4 times a year, such as 5 times a year, such as 6 times a year, such as 7 times a year, such as 8 times a year, such as 9 times a year, such as 10 times a year, such as 11 times a year, such as 12 times a year, such as every month, such as 2 times a month, such as 3 times a month, such as 4 times a month, such as every week, such as 2 times a week, such as 3 times a week, such as 4 times a week, such as 5 times a week, such as 6 times a week, such as 7 times a week, such as every day, such as 2 times a day, such as 3 times a day, such as 4 times a day, such as 5 times a day, such as 6 times a day, such as 7 times a day, such as 8 times a day, such as 9 times a day, such as 10 times a day, such as 11 times a day, such as 12 times a day, such as 13 times a day, such as 14 times a day, such as 15 times a day, such as 16 times a day, such as 17 times a day, such as 18 times a day, such as 19 times a day, such as 20 times a day, such as 21 times a day, such as 22 times a day, such as 23 times a day, such as 24 times a day, such as every hour, such as 2 times a hour, such as 3 times a hour, such as 4 times a hour, such as 5 times an hour, such as 6 times an hour, such as 7 times a hour, such as 8 times a hour, such as 9 times a hour, such as 10 times a hour, such as every minute, such as every 30 seconds.

The monitoring may be performed by a non-medically trained or qualified person, optionally in a location away from a hospital, such as in the persons own home or at any other location. It is an advantage of the present invention that the method according to the invention, including the monitoring according to the invention can be performed without the need for assistance by medically trained or qualified personnel. It is a further advantage of the present invention that the method according to the invention, such as the monitoring according to the invention, can be carried out at any location, such as away from a hospital.

In yet a further aspect there is provided a method for diagnosing a clinical condition, said method comprising the steps of

i) detecting the presence in a sample of ERAC according to the invention, or quantifying in a sample the amount of ERAC according to the invention, or profiling according to the invention, or monitoring the presence of ERAC according to the invention, and
ii) diagnosing said clinical condition.

In still a further aspect there is provided a method for prognosing the outcome or development or relapse or remittance or progress of a clinical condition in an individual, said method comprising the steps of

i) detecting the presence in a sample of ERAC according to the invention, or quantifying in a sample the amount of ERAC according to the invention, or profiling according to the invention, or monitoring the presence of ERAC according to the invention, and
ii) determining the prognosis of said individual.

In yet a further aspect there is provided a method for treatment of a clinical condition, said method comprising the step of

i) reducing in an individual in need thereof the amount of ERAC present in a body fluid of said individual.

In still a further aspect there is provided a method for treatment of a clinical condition, said method comprising the step of

i) administering to an individual in need thereof a compound capable of competing with ERAC for binding to a receptor.

Preferably said receptor is a receptor to which ERAC binds, more preferably said receptor is the receptor for advanced glycation end products (RAGE)

In yet a further aspect there is provided a method for treatment of a clinical condition, said method comprising the step of

i) monitoring the presence of ERAC according to the invention, and
ii) treating the clinical condition according to the invention.

In still a further aspect there is provided a method for treatment of a clinical condition, said method comprising the step of

i) performing a diagnosis of a clinical condition associated with ERAC, and
ii) treating said clinical condition according to the invention.

In yet a further aspect there is provided a method for treatment of a clinical condition, said method comprising the step of

iii) performing a prognosis according to the invention, and
iv) treating the clinical condition according to the invention.

In still a further aspect there is provided a method of treatment according to the invention, wherein said method is combined with treatment of said clinical condition involving the administration of an anti-inflammatory agent.

A clinical condition according to the invention may be chronic and acute inflammatory conditions, auto-immune diseases, cancer, kidney diseases or mal-functions, cardiovascular disease or infection.

The clinical condition may be selected from the group consisting of the infectious conditions Actinomycosis, Adenovirus-infections, Antrax, Bacterial dysentery, Botulism, Brucellosis (Bang's disease), caused by e.g. B. melitensis and B. suis, Candidiasis, Cellulitis, Chancroid, Cholera, Coccidioidomycosis, Acute afebril, Conjunctivitis, Cystitis, Dermatophytosis, Bacteriel Endocarditis, Epiglottitis, Erysipelas, Erysipeloid, Gastroenteritis, Genital herpes, Glandulae, Gonorrhea, Hepatitis, Viral Hepatitis, Histoplasmose, Impetigo, Malaria, Mononucleosis, Influenza, Legionaires disease, Leptospirosis, Lyme disease, Melioidosis, Meningitis, Nocardiosis Nocardia asteroides, Nongonococcal urethritis, Pinta, Pneumococcal lung disease, Poliomyelitis, Primary lung infection, Pseudomembranious enterocolitis, antibiotic-associated Puerperal sepsis, Rabies, Relaps-fever, Rheumatic fever, Rocky Mountain spotted-fever, Rubella, Rubeola, Staphylococcal scalded skin syndrome, Streptococcal pharyngitis (strep throat), Syphilis, Tetanus, Toxic shock syndrome, Toxoplasmose, Tuberculosis, Tularemia, Typhoid fever, Typhus, Vaginitis, Varicella, Verrucae, Pertussis, Framboesia (Yaws), and Yellow fever.

The clinical condition may be selected from the group consisting of asthma, autoimmune disease, chronic inflammation, chronic prostatitis, glomerulonephritis, graft versus host disease, host versus graft disease, hypersensitivity, inflammatory bowel disease, inflammatory myopathy, pelvic inflammatory disease, pre-eclampsia, reperfusion injury, rheumatoid arthritis, Sjögren's syndrome, transplant rejection, and vasculitis.

The clinical condition may be selected from the group consisting of aneurysm, angina pectoris, atherosclerosis, cerebral haemorrhage, cerebro vascular disease, congestive heart failure, coronary artery disease, hypertension, myocardial infarction, stable angina pectoris, stroke, and unstable angina pectoris.

In one aspect of the invention the clinical condition may preferably be rheumatoid arthritis. In another aspect, the clinical condition may preferably be Sjögren's syndrome. In yet another aspect, the clinical condition may preferably be pre-eclampsia.

In still a further aspect there is provided a computer readable medium comprising instructions for carrying out the method of quantifying according to the invention.

In yet a further aspect there is provided an automated system suitable for carrying out the method of quantifying according to the invention, comprising, in combination:

i) a database capable of including a plurality of digital images,
ii) a software module for analyzing a plurality of pixels from a digital image,
iii) a control module comprising instructions for carrying out the method of quantifying ERAC according to the invention.

In still a further aspect there is provided a software program loadable into the memory of a computer, said program comprising instructions for carrying out the method of quantifying ERAC according to the invention.

The invention in a still further embodiment comprises measurements of ERAC in the absence of measurement of native S100A12. In one embodiment the invention is directed only to the detection of ERAC and therefore the invention does not comprise detection of native S100A12 oligomers, which dissociate when treated with EDTA.

The present invention discloses a polypeptide complex comprising a plurality of S100A12 monomers or oligomers, wherein a monomer of S100A12 has the amino acid sequence SEQ ID NO:1:

### TKLEEHLEGIVNIFHQYSVR KGHFDTLSKGELKQLLTKEL

### ANTIKNIKDKAVIDEIFQGL DANQDEQVDFQEFISLVAIA

### LKAAHYHTHKE,

or is substantially identical to SEQ ID NO:1.

The present invention is also directed to methods for making or using polypeptide complexes according to the present invention.

### DEFINITIONS

As used everywhere herein, the term “a”, “an” or “the” is meant to be one or more, i.e. at least one.

Antibodies: As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Antibodies can derive from multiple species. For example, antibodies include rodent (such as mouse and rat), rabbit, sheep, camel, and human antibodies. Antibodies can also include chimeric antibodies, which join variable regions from one species to constant regions from another species. Likewise, antibodies can be humanized, that is constructed by recombinant DNA technology to produce immunoglobulins which have human framework regions from one species combined with complementarity determining regions (CDR's) from a another species' immunoglobulin. The antibody can be monoclonal or polyclonal. Antibodies can be divided into isotypes (IgA, IgG, IgM, IgD, IgE, IgG1, IgG2, IgG3, IgG4, IgA1, IgA2, IgM1, IgM2)

Antibodies: In another embodiment the term “antibody” refers to an intact antibody, or a fragment of an antibody that competes with the intact antibody for antigen binding. In certain embodiments, antibody fragments are produced by recombinant DNA techniques. In certain embodiments, antibody fragments are produced by enzymatic or chemical cleavage of intact antibodies. Exemplary antibody fragments include, but are not limited to, Fab, Fab′, F(ab′)2, Fv, and scFv. Exemplary antibody fragments also include, but are not limited to, domain antibodies, nanobodies, minibodies ((scFv-C.sub.H3).sub.2), maxibodies ((scFv-C.sub.H2-C.sub.H3).sub.2), diabodies (noncovalent dimer of scFv).

Antibody fragment: An antibody fragment is a portion of an antibody such as F(ab′)₂, F(ab)₂, Fab′, Fab, and the like. Regardless of structure, an antibody fragment binds with the same antigen that is recognized by the intact antibody. For example, an anti-(polypeptide according to the present invention) monoclonal antibody fragment binds an epitope of a polypeptide according to the present invention. The term antibody fragment also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

Bioluminescent: Bioluminescence, as used herein, is the production and emission of light by a living organism as the result of a chemical reaction during which chemical energy is converted to light energy.

Chemiluminescent: Chemiluminescence, as used herein, is the emission of light (luminescence) without emission of heat as the result of a chemical reaction.

Chimeric antibody: A “chimeric antibody” is a recombinant protein that contains the variable domains and complementary determining regions derived from a rodent antibody, while the remainder of the antibody molecule is derived from a human antibody.

Chromophore: A chromophore, as used herein, is the part of a visibly coloured molecule responsible for light absorption over a range of wavelengths thus giving rise to the colour. By extension the term can be applied to uv or it absorbing parts of molecules.

Covalent binding: The term covalent binding is used herein to describe a form of chemical bonding that is characterized by the sharing of pairs of electrons between atoms. Attraction-to-repulsion stability that forms between atoms when they share electrons is known as covalent bonding.

Diagnosis: The act or process of identifying or determining the nature and cause of a disease or injury through evaluation

Detectable label: A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

Fluorescent: the term fluorescent as used herein is to have the ability to emit light of a certain wavelength when activated by light of another wavelength.

Fluorochromes: fluorochrome, as used herein, is any fluorescent compound used as a dye to mark e.g. protein with a fluorescent label.

Fluorophore: A fluorophore, as used herein, is a component of a molecule which causes a molecule to be fluorescent.

Humanized antibodies: “Humanized antibodies” are recombinant proteins in which murine complementarity determining regions of a monoclonal antibody have been transferred from heavy and light variable chains of the murine immunoglobulin into a human variable domain.

Label: Label herein is used interchangeable with labeling molecule. Label as described herein is an identifiable substance that is detectable in an assay and that can be attached to a molecule creating a labeled molecule. The behavior of the labeled molecule can then be studied.

Labelling: Labelling herein means attachment of a label to a molecule.

Monoclonal antibodies: Monoclonal antibodies, as used herein, are antibodies that are identical because they were produced by one type of immune cell and are all clones of a single parent cell.

Monovalent antibodies: The antibodies in the present invention can be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. In vitro methods are also suitable for preparing monovalent antibodies. Digestion of antibodies to produce fragments thereof, particularly, Fab fragments, can be accomplished using routine techniques known in the art.

Oligomer: An oligomer is a compound consisting of a limited number of repeating structural units, i.e. monomers, usually from 2 to 20, connected by covalent chemical bonds. An oligomer may for instance consist of two monomers connected by one or more covalent chemical bonds.

Peptide or protein: Any molecule composed of at least two amino acids. Peptide normally refers to smaller molecules of up to around 30 amino acids and protein to larger molecules containing more amino acids.

Polyclonal antibodies: Polyclonal antibodies as used herein are antibodies that are derived from different B-cell lines. They are a mixture of immunoglobulin molecules secreted against a specific antigen, each recognising a different epitope.

Polymer: Polymer as used herein is defined as a compound composed of any number of repeating structural units, or monomers, connected by covalent chemical bonds.

Polypeptide: Peptides are the family of short molecules formed from the linking, in a defined order, of various α-amino acids. The link between one amino acid residue and the next is an amide bond and is sometimes referred to as a peptide bond. Longer peptides are referred to as proteins or polypeptide.

Prognosis: The term “prognosis” refers in general to a prediction of the course of a clinical condition in an individual, e.g. the outlook for the cure of the patient. A prognosis may preferably aim at determining the likely or possible outcome or progress or development or relapse or remittance of a clinical condition in an individual.

Radioactivity: Radioactive decay is the process in which an unstable atomic nucleus loses energy by emitting radiation in the form of particles or electromagnetic waves.

Sample: In the present context the word sample is used interchangeably with the word specimen.

Standard: A standard is an illustration of the result of one or more measurements of samples with known concentrations of the substance to be detected. The standard may be in the form of a standard curve. A standard curve is a plurality of measurements of different known concentrations of the substance to be detected. A standard curve is usually depicted as a graph or diagram or chart. On a lateral flow device a standard may be in the form of a number of dots or bands or lines or zones containing different known concentrations of the substance to be detected bound to labelled targeting species. For instance, the standard may be in the form of a plurality of lines comprising different known concentrations of ERAC bound to a labelled first targeting species. The standard can for instance be used to compare the measurement made on a sample of unknown concentration to the different standard lines indicated, thereby determining the approximate concentration of the measured substance in the sample. A standard, such as a standard curve, according to the present invention may also be in digital or digitally readable form, such as in the form of a barcode or chip. For instance, a standard or standard curve may be present as a barcode on a kit according to the invention.

Treatment: Treatment can be performed in several different ways, including curative, ameliorating and as prophylaxis. Curative treatment generally aims at curing a clinical condition, such as a disease or an infection, which is already present in the treated individual. Ameliorating treatment generally means treating in order to improve in an individual an existing clinical condition. Prophylactic treatment generally aims at preventing a clinical condition.

### DESCRIPTION OF DRAWINGS

FIG. 1 shows results from a GPC experiment on a High-Load Superdex-75 column from Pharmacia, Sweden. Column size is 1.6×60 cm. The buffer is tris-buffered saline, 2 mM calcium chloride, pH 8.0. The sample is 0.3 ml normal human serum. The flow rate is 1 ml/min.

FIG. 2 shows results from a GPC experiment on a High-Load Superdex-75 column from Pharmacia, Sweden. Column size is 1.6×60 cm. The buffer is tris-buffered saline with 5 mM EDTA. The sample is 0.3 ml normal human serum. The flow rate is 1 ml/min.

FIG. 3 shows results from a GPC experiment on a High-Load Superdex-75 column from Pharmacia, Sweden. Column size is 1.6×60 cm. The buffer is tris-buffered saline with 5 mM EDTA, pH 8.0. The sample is 0.3 ml rheumatoid arthritis patient serum. The flow rate is 1 ml/min.

FIG. 4 shows results from a DEAE anion exchange chromatography experiment run with a stepwise sodium chloride gradient. The sample is 0.3 ml human rheumatoid arthritis patient serum. The column is a DEAE-sepharose Fast Flow (Pharmacia, Sweden) size 12×18 mm. The buffer is 25 MM sodium barbital, pH 8.8. The sample is 0.3 ml rheumatoid arthritis patient serum containing some ERAC but mostly normal S100A12. The S100A12 eluted with 200 mM sodium chloride represents ERAC.

FIG. 5 shows a typical standard curve for S100A12 ELISA with concentration on the X-axis and optical density (OD) on the Y-axis.

FIG. 6

FIG. 6 is a picture showing an embodiment of a kit according to the invention in the form of lateral flow devices. All four lateral flow devices are suited for the measurement of ERAC in a sample. The samples applied to the four different lateral flow devices are as indicated in the table below.

| | |
|---|---|
| Non-EDTA treated ERAC | Non-EDTA treated ERAC |
| positive sample | negative sample |
| EDTA treated ERAC positive | EDTA treated ERAC negative |
| sample | sample |

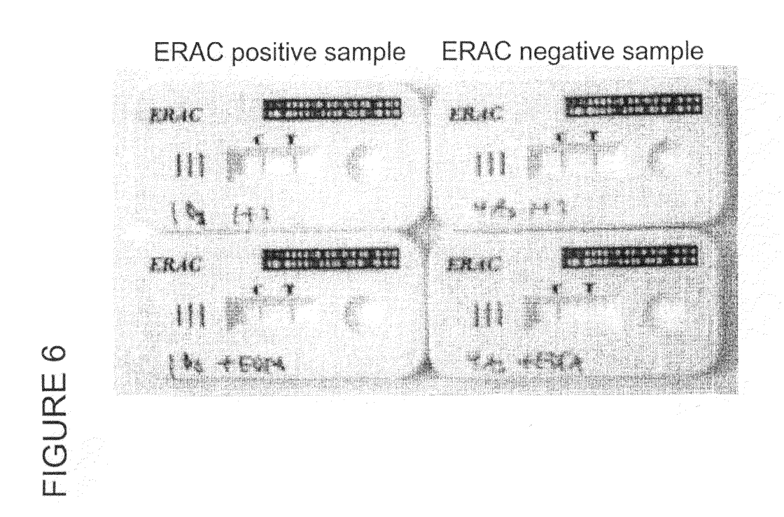

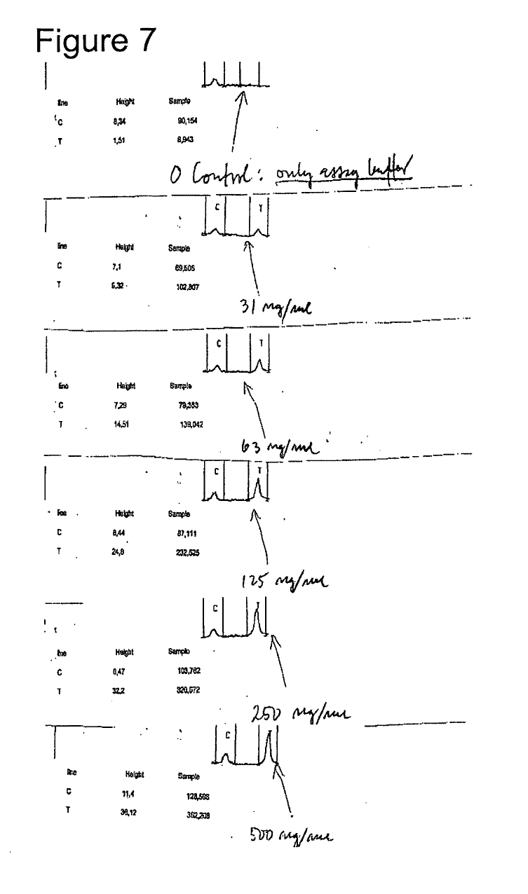

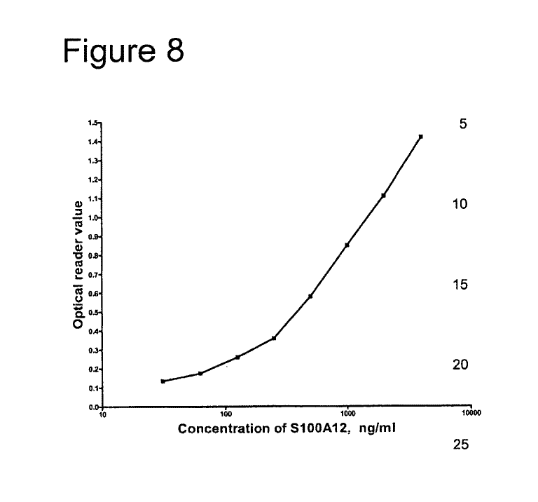

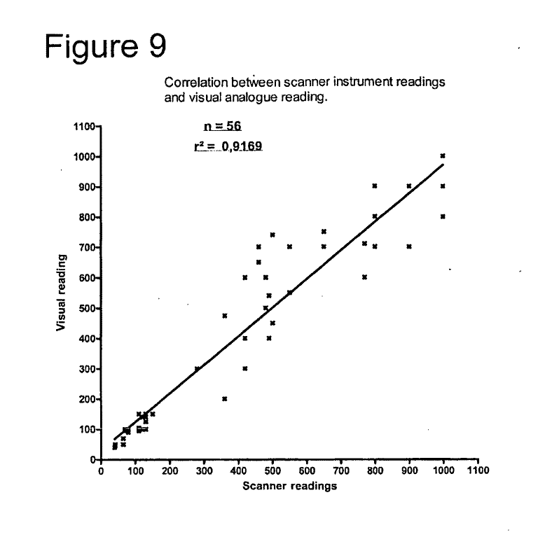

As can be seen in the picture of FIG. 6, when the sample is treated with EDTA only the ERAC containing samples (ERAC positive samples) give rise to a positive test result, i.e. a clear line at the test zone (“T”). In all instances, the control zone (“C”) is positive as should always be the case when using a lateral flow device with a control zone. The application zone of the lateral flow device can be seen as a circular opening to the right of the test zone. This is where sample has been applied to the device.

FIG. 7

FIG. 7 shows a graph of quantitative measurement of ERAC in a sample. The staining intensity of the test line on the ERAC lateral flow test described above can for instance be determined by photometric instruments. FIG. 7 shows a typical scanning curve when a LFT test strip has been scanned. As shown, the control line gives peaks of equal size irrespective of the protein concentrations (0 to 500 ng/ml), while the test line size increases with increasing concentration of ERAC in the sample.

FIG. 8

FIG. 8 shows a standard curve obtained when samples with ERAC concentrations between 50 and 2000 ng/ml were tested and read by use of a scanner.

FIG. 9

FIG. 9 shows the correlation between scanner instrument readings and visual analogue reading of lateral flow devices according to the invention. On the x-axis are plotted the scanner readings and on the y-axis are plotted the visual analogue readings.

### DETAILED DESCRIPTION OF THE INVENTION

For the purpose of studying of patients with rheumatoid arthritis or radiation damage of the bowel, an enzyme immunoassay (ELISA) for human A12 was developed. The normal range was determined by assaying samples from healthy individuals. It was found that the assay results were strongly dependent on the concentration of calcium in the sample; in fact very little, if any, A12 could be detected in normal plasma since calcium binding chemical like EDTA or citrate is used to prevent the blood from clotting. Since it has been shown that A12 can form dimers and oligomers in the presence of calcium, we decided to check for such complexes in serum by running samples of serum with or without the addition of EDTA, on a gel permeation chromatography column. That method can separate protein molecules according to molecular weight. We could confirm that in samples from healthy individuals A12 is present as monomers or oligomers with molecular weight up to about 800 kDa if the chromatography buffer contains calcium, for instance 2 mM calcium chloride. In contrast, if the chromatography buffer contains EDTA, for instance 2 g/liter, A12 elutes as a monomer corresponding to a molecular weight of about 10 kDa. It was very surprisingly found that serum from some patients and a small proportion of seemingly healthy, regular blood donors contained a high molecular weight A12 even if EDTA was added. Accordingly, this substance was called “ERAC”, which is an abbreviation for EDTA Resistant A12 Complex.

The present invention concerns the fact that in humans, the leukocyte derived protein S100A12 may occur in serum in a high molecular weight form, corresponding to 500 kDa or higher, that resists dissociation into monomers in the presence of EDTA. ERAC has been found in about 50% of patients with rheumatoid arthritis. In contrast, ERAC was present in only two out of 150 regular blood donors at the Blood Bank, University Hospital of Bergen, Norway. Despite being seemingly healthy, these two blood donors had serum A12 concentrations about 13 micrograms per litre which is ten times the upper reference limit.

The present invention directed to a high molecular weight form of the leukocyte derived protein S100A12 (A12) in a high proportion, about 50%, of patients with rheumatoid arthritis or similar diseases and in a small proportion, about 1.5%, of healthy adults.

During an evaluation of a newly established enzyme immunoassay (ELISA) for A12 and its use in different patients groups it was found that the assay results depended of the presence or absence of calcium. Levels of A12 in plasma, typically from EDTA anticoagulated blood, were very low or undetectable. It was hypothesized that the antibody used reacted with a single antigenic epitope on A12, so that a positive reaction in the ELISA would require complex formation between two or more A12 monomers, i.e. the presence of A12 dimers, oligomers or polymers. Since we found that the ELISA readings increased in proportion with the concentration of calcium, we wanted to test whether the assayed levels correlated with higher proportion of dimers/oligomers. For this purpose we ran serum samples on a gel permeation chromatography (GPC) column, High Load Superdex-75 (Pharmacia, Sweden) equilibrated with tris buffered saline with 2 mM calcium chloride (pH 8.0).

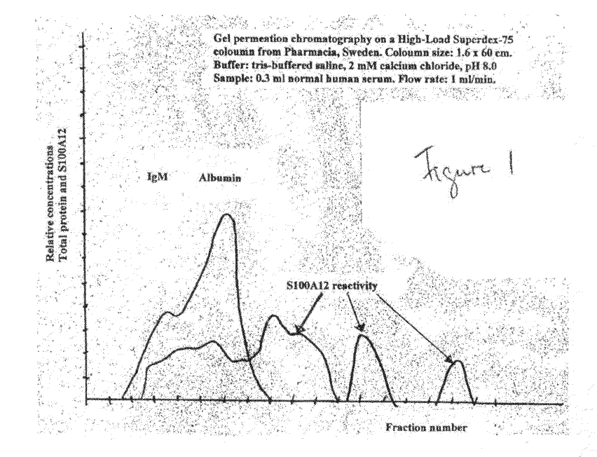

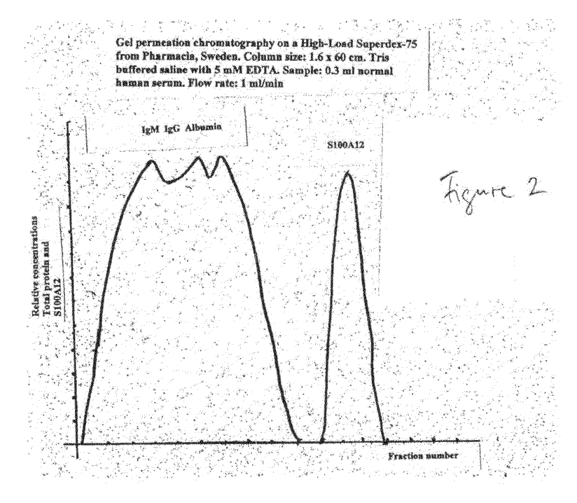

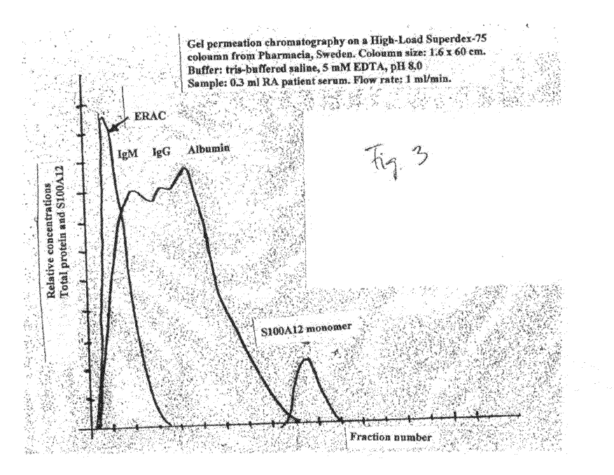

FIG. 1 shows that in normal serum A12 was detected in at least six fractions corresponding to molecular weights from 10 to about 500 kDa. In contrast, if the buffer contained no calcium, but 5 mM EDTA, A12 eluted in a single fraction corresponding to a molecular weight of 10 kDa, i.e. like the monomer. This is shown in FIG. 2. Characteristically, this fraction gave no reaction in the ELISA unless calcium chloride was added to overcome the binding capacity of the EDTA. On one occasion serum from a rheumatoid arthritis (RA) patient was run with the EDTA containing buffer, and unintentionally no calcium was added to the fraction before running the ELISA. To our big surprise a strong reactivity was found in an early fraction, i.e. corresponding to a much larger molecular weight, than we had ever seen before; neither has such a finding been published. It was first thought to be an artifact, but the pattern, see FIG. 3, was confirmed by many repeat runs. We therefore concluded that some serum samples contain a high molecular weight substance with A12; in contrast to other A12 complexes (dimers, oligomers/polymers), it does not dissociate in the presence of EDTA.

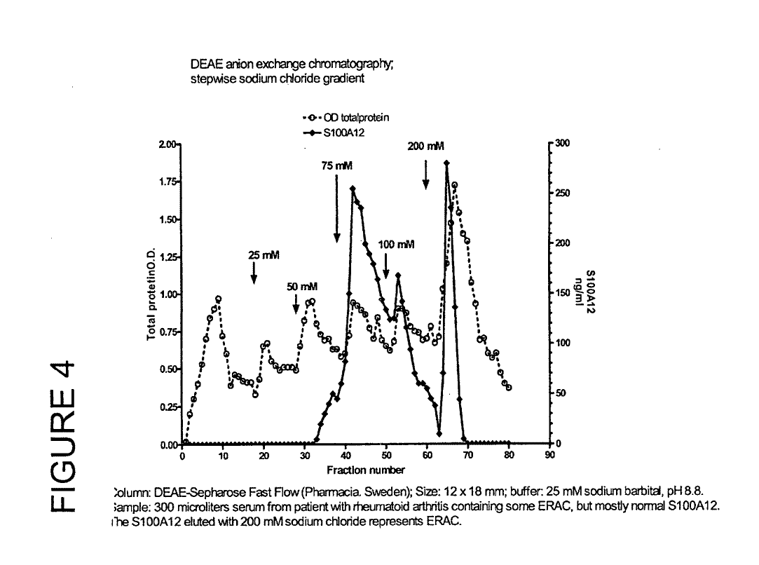

Subsequently sera from many RA patients and some healthy control were run on the GPC column to check for ERAC. We found that 9 out of 13 RA patients and one out of two patients with Sjögrens syndrome had ERAC. As shown in FIG. 4, ERAC may be present together with various amounts of monomeric A12. From the patient's records it was clear that ERAC positivity correlated with the presence of extra-articular pathology, typically arteriosclerotic disease. To our big surprise, ERAC was also found in two healthy, regular blood donors which might suggest subclinical pathology. All the ERAC positive individuals had high A12 levels in serum. Samples taken from two individuals shortly after an exhaustive long distance run did not show ERAC even if the A12 levels were 50 to 80 times the upper reference level.

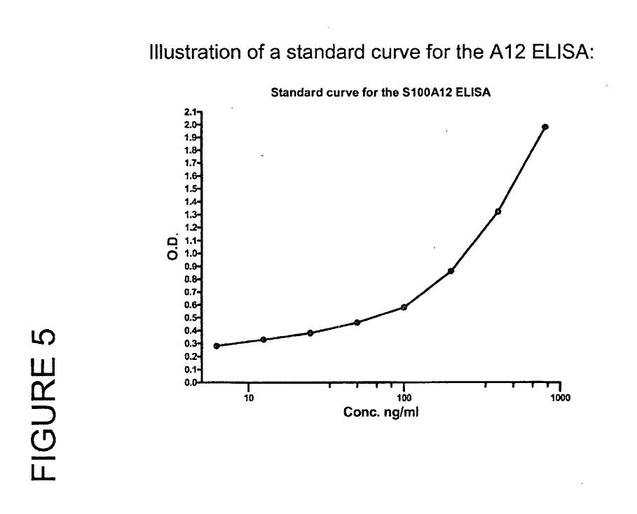

ERAC is also characterized by its binding to a weak anion exchange material; when ERAC fractions from the GPC are applied on a DEAE-Sepharose Fast Flow column equilibrated with a 25 mM sodium barbital buffer, pH 8.8, ERAC will bind to the anion exchanger and can be eluted with 200 mM sodium chloride, see FIG. 5.

It is well known that A12 is capable of inducing inflammation, and since that protein is releases from neutrophil granulocytes at sites of inflammation, for instance joints in RA patients, one can hypothesize that large complexes like ERAC may remain in tissues and induce or maintain inflammation.

It has been commonly accepted that atheromatosis, the pathological process causing for instance coronary heart disease is an inflammatory disease. Again, ERAC may play a pathogenetic role. The finding of ERAC in seemingly healthy individuals may be a marker of subclinical theromatosis which could be useful with regard to introduction of early preventive measures.

Accumulation of large amounts of high molecular weight complexes of calprotectin (a heterotrimer of S100A8 and S100A9) which is closely related to A12 gives rise to the “Calprotectin syndrome” (References 6 and 7) characterized by multi-organ pathology including arthritis. It is an attractive hypothesis that this syndrome as well as disease related to ERAC may be some kind of prion disease. By use of in-vitro replication techniques for prions (Reference 8) it is possible to test whether added, normal A12 will bind to ERAC.

If ERAC is capable of inducing or maintaining chronic inflammation, therapies may aim at removing or inactivating ERAC.

Assays for ERAC may be useful both for research and clinical purposes; ERAC may be used to monitor pathological processes where A12 are involved, for instance during trials of new drugs, to monitor disease activity and response to treatment. ERAC assays may be based upon the finding that only ERAC gives a positive signal in ELISA, and probably other immunoassays, in the presence of EDTA.

Variants of ERAC and of SEQ ID NO:1

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])×(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative or variant. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g. SEQ ID NO:1) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

The present invention is also directed to variant polypeptides having one or more conservative amino acid substitution(s) and polynucleotides encoding polypeptides having one or more conservative amino acid substitution(s), as compared with the amino acid sequence of SEQ ID NO:1. Variants of SEQ ID NO:1 include sequences wherein e.g. an alkyl amino acid is substituted for an alkyl amino acid, wherein an aromatic amino acid is substituted for an aromatic amino acid, wherein a sulfur-containing amino acid is substituted for a sulfur-containing amino acid in, wherein a hydroxy-containing amino acid is substituted for a hydroxy-containing amino acid, wherein an acidic amino acid is substituted for an acidic amino acid, wherein a basic amino acid is substituted for a basic amino acid, or wherein a dibasic monocarboxylic amino acid is substituted for a dibasic monocarboxylic amino acid.

Among the common amino acids, for example, a “conservative amino acid substitution” can also be illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Particular variants of SEQ ID NO:1 are characterized by having at least 70%, at least 80%, at least 85%, at least 90%, at least 95% or greater than 95% sequence identity to SEQ ID NO:1, e.g. when the variation in amino acid sequence is due to one or more conservative amino acid substitutions. Preferably, the polypeptide according to the present invention is at least 70% identical to SEQ ID NO:1, such as at least 75% identical, for example at least 80%, such as at least 85%, for example at least 90%, such as at least 95%.

Production of antibodies specific for polypeptides according to the present invention Antibodies to ERAC, or a fragment thereof, can be obtained by using ERAC according to the present invention isolated from a natural source. Particularly useful antibodies “bind specifically” with ERAC. ERAC and fragments thereof are in the following collectively referred to as polypeptides according to the present invention.

Antibodies are considered to be specifically binding if the antibodies exhibit at least one of the following two properties: (1) antibodies bind to a polypeptide according to the present invention with a threshold level of binding activity, and (2) antibodies do not significantly cross-react with polypeptides which are related to a polypeptide according to the present invention as defined herein below.

With regard to the first characteristic, antibodies specifically bind if they bind to a polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660 (1949)). With regard to the second characteristic, antibodies do not significantly cross-react with related polypeptide molecules, for example, if they detect polypeptides according to the present invention, but do not detect known polypeptides applied in similar or identical amounts in a standard Western blot analysis.

Antibodies can be produced using antigenic epitope-bearing peptides or polypeptides according to the present invention. Antigenic, epitope-bearing peptides and polypeptides of the present invention preferably contain a sequence of at least four, or between 15 to about 30 amino acids contained within SEQ ID NO:1, or a fragment thereof. However, peptides or polypeptides comprising a larger portion of SEQ ID NO:1, such as a sequence containing from 30 to 50 amino acids, or any length up to and including the entire amino acid sequence of a polypeptide according to the invention (SEQ ID NO:1 and variants thereof), also are useful for inducing antibodies that bind with polypeptides according to the present invention. It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may be also be desirable for antibody production.

As an illustration, potential antigenic sites in polypeptides according to the present invention can be identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Conformation, Fasman (ed.), pages 549 586 (Plenum Press 1990), and Garnier-Robson, Garnier et al., J. Mol. Biol. 120:97 (1978) (Chou-Fasman parameters: conformation table=64 proteins; .alpha. region threshold=103; .beta. region threshold=105; Garnier-Robson parameters: .alpha. and .beta. decision constants=0). In the sixth subroutine, flexibility parameters and hydropathy/solvent accessibility factors were combined to determine a surface contour value, designated as the “antigenic index.” Finally, a peak broadening function was applied to the antigenic index, which broadens major surface peaks by adding 20, 40, 60, or 80% of the respective peak value to account for additional free energy derived from the mobility of surface regions relative to interior regions. This calculation was not applied, however, to any major peak that resides in a helical region, since helical regions tend to be less flexible.

Polyclonal antibodies to ERAC can be prepared using methods well-known to those of skill in the art. See, for example, Green et al., “Production of Polyclonal Antisera,” in Immunochemical Protocols (Manson, ed.), pages 1 to 5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a polypeptide can be increased through the use of an adjuvant, such as alum (aluminum hydroxide) or Freund's complete or incomplete adjuvant. Polypeptides useful for immunization also include fusion polypeptides, such as fusions of ERAC, or a portion thereof, with an immunoglobulin polypeptide, or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an antibody specific for a polypeptides according to the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal antibodies specific for a polypeptides according to the present invention can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1 2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an antibody specific for polypeptides according to the present invention of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1 2.7.12 and pages 2.9.1 2.9.3; Baines et al., “Purification of Immunoglobulin G (IgG),” in Methods in Molecular Biology, Vol. 10, pages 79 104 (The Humana Press, Inc. 1992)).

For particular uses, it may be desirable to prepare fragments of antibodies specific for polypeptides according to the present invention. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an F_{c} fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al. and Coligan, both in Methods in Enzymology Vol. 1, (Academic Press 1967).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to polypeptide in vitro, and selecting antibody display libraries in phage or similar vectors (for instance, through use of immobilized or labeled protein or peptide). Genes encoding polypeptides having potential polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the sequences disclosed herein to identify proteins which bind to.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an antibody specific for a polypeptide according to the present invention may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399 434 (John Wiley & Sons, Inc. 1996), and by Queen et al., U.S. Pat. No. 5,693,762 (1997).

Polyclonal anti-idiotype antibodies can be prepared by immunizing animals with antibodies or antibody fragments specific for a polypeptide according to the present invention, using standard techniques. See, for example, Green et al., “Production of Polyclonal Antisera,” in Methods In Molecular Biology: Immunochemical Protocols, Manson (ed.), pages 1 to 12 (Humana Press 1992). Also, see Coligan at pages 241 to 247. Alternatively, monoclonal anti-idiotype antibodies can be prepared using antibodies or antibody fragments specific for a polypeptide according to the present invention as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

ERAC and Kits and Methods for Detection of ERAC

S100A12 is a calcium-binding protein predominantly found in neutrophil granulocytes and monocytes. The protein has intra- and extra-cellular functions, particularly by inducing inflammation by binding to the receptor, RAGE (Receptor for Advanced Glycation End Products), which can be found on e.g. endothelial cells. One hypothesis regarding S100A12 is that it is the entity of six molecules that interact with the receptor. However, this hypothesis may be extended by our new finding of a yet larger complex (ERAC), possibly as a pathological complex found in rheumatoid arthritis (RA) patients.

The findings related to ERAC (EDTA Resistant S100A12 Complexes) indicate that ERAC are found in high-molecular weight (typically 100-400 kDa) fractions of gel permeation chromatography. One important feature of ERAC is the resistance to dissolve into S100A12 monomers in the presence of EDTA. We hypothesise that substances (endogenous, exogenous) can influence ERAC to dissolve into smaller molecular weight complexes. Pharmaceutical treatment of atherosclerosis may reduce the risk of ischemic disease. The hypothesis is that ERAC bind to endothelial cell receptors, inducing a pathological, sustained pro-inflammatory signal. Pharmaceutical treatment that might dissolve ERAC, might shorten the duration of the pathological prolonged binding to the receptor. Possible pharmaceuticals in this respect could be anti-inflammatory medicines e.g. acetyl-salicylic acid and statins. Another mechanism, by which pharmaceuticals could interfere in a hypothesised pathological binding to receptors, could be RAGE antagonists, analogue to the mechanisms of TNF-α antagonist.

The present invention provides in an embodiment a method for detection of ERAC in a specimen or sample, wherein said specimen, optionally treated to remove undesired components, is contacted with a kit comprising a targeting species, preferably an antibody, directed against ERAC. The contacting results in the case of an antibody being used in the formation of immuno-complexes with ERAC antigens.

The kit is separated from the specimen; said separated kit is contacted with a mobile solid phase comprising a polymeric carrier molecule according to the invention having conjugated thereto a predetermined targeting species such as an antibody. The antibody results in the binding of said polymeric carrier molecules according to the invention to said immuno-complexes; the kit is subsequently separated from said mobile solid phase; and the presence of polymeric carrier molecules according to the invention bound to said kit is detected, whereby the presence of ERAC in said specimen is detected or determined.

Also, the invention provides an ERAC detection kit which comprises as individual components: (a) an solid support having conjugated thereon a targeting species, preferably an antibody capable of forming immuno-complexes with antigens characteristic of ERAC; and (b) a mobile solid phase consisting of dispersed polymeric carrier molecules according to the invention having conjugated thereto said targeting species, or a different target species, preferably an antibody, characteristic of ERAC.

A specimen which may comprise ERAC is in one embodiment exposed to a kit which is coated at least in one location with a targeting species which will form complexes with the antigens of ERAC.

The kit is in one embodiment separated from the specimen, such as by washing the specimen off the kit, and the separated kit is then contacted with a mobile solid phase of dispersed polymeric carrier molecules according to the invention comprising the same or different targeting species, preferably an antibody. If immuno-complexes of ERAC have formed on the solid support of the kit, the polymeric carrier molecules according to the invention will be bound to such complexes.

The unbound polymeric carrier molecules according to the invention of the mobile solid phase then are removed, such as by washing, and the kit is examined to determine the presence of polymeric carrier molecules according to the invention bound to the kit. These may be visually detected in some cases, for example when the polymeric carrier molecules according to the invention have been initially stained or dyed. Microscopic examination may be employed. The use of tracers or labels for the polymeric carrier molecules according to the invention enables the use of other detection methods as described herein below in more detail.

By this means, the presence or absence of bound polymeric carrier molecules according to the invention enables detection of the presence or absence of ERAC, and an evaluation of the quantity of bound polymeric carrier molecules according to the invention enables determination of the quantity of ERAC in the specimen, for example by comparison with standard results or a standard curve obtained by assaying samples with known concentrations of ERAC.

The kit used in the present invention may be employed in a variety of forms or structures. The solid phase has a location where a targeting species, preferably an antibody, can bind or associate, and the formation of such a solid phase with said targeting species, preferably an antibody, enables contacting a specimen and other materials used in the method of the invention. Preferred specimens are body fluid samples as described herein.

The kit is best formed in a way which enables simple manipulation for easy contact with the specimen and other reagents. For this purpose, the kit may form at least part of a dipstick, syringe, tube or container.

The specimen and other reagents can be drawn in and ejected from a syringe, caused to flow through a tube, or deposited in a container such as a test tube shaped container. In such devices, the kit can form the whole of the device, or part of it, where, in the case of a syringe, tube or container, the part formed of the kit will at least be exposed at the inside of the device to permit contact with specimen and reagents. Targeting species, preferably an antibody, is preferably concentrated at one location of the solid support of said kit, to be exposed to the specimen.

One more preferred form of the kit is a dipstick. In such a dipstick, it is further preferred that the kit should be included at least one end, and that the targeting species, preferably an antibody, conjugated on the solid support of said kit should be concentrated at the end of the dipstick. The kit can however comprise the entire dipstick, with the targeting species, preferably an antibody, concentrated at one end, or in more than one location.

The dipstick may be entirely formed from the kit, at one end of which has been conjugated a coating of targeting species, preferably an antibody. In another embodiment the dipstick has a kit one end of which is adhered to a body portion. A coating of targeting species, preferably an antibody, is conjugated to the kit. In yet another embodiment the kit entirely forms a tubular container into which a specimen can be placed. Coatings of targeting species, preferably an antibody, are located near the bottom of the container and are concentrated in one or more locations.

The solid support of said kit is composed of any material onto which the desired targeting species, preferably an antibody, can be effectively bound. For covalent binding with antibody protein, the solid support material can be chosen to contain a functional carboxyl surface, with use of a water-soluble carbodiimide as a conjugation reagent. A preferred material is acrylic resin, which has a carboxylated surface that enables binding the desired targeting species, preferably an antibody, by conjugation. For materials with amino surface groups, reactive carboxyl intermediates can be prepared by reacting with succinic anhydride. A variety of inorganic supports, typically glass, can also be prepared for covalent coupling with targeting species, preferably an antibody.

Solid support materials capable of binding targeting species, preferably an antibody, are selected from materials which do not cause serious interference with the method steps. Solid support material may for instance be selected from the following materials:

### Whatman GF/D

### Whatman F147-11

### Whatman GF/AVA

### Whatman 147-02

### Whatman GF/DFA

### Whatman F147-09

### Whatman F075-17*

### Millipore Rapid Q24*

### Millipore Rapid Q27

### Ahlstrom A142

### Millipore Hi-Flow Plus HF07504

### Millipore Hi-Flow Plus HF09004

### Millipore Hi-Flow Plus HF12004

### Millipore Hi-Flow Plus HF13504

### Millipore Hi-Flow Plus HF18004

### Sartorius Unisart CN40

### Sartorius Unisart CN90

### Sartorius Unisart CN200*

The presence of non-specific agglutinators in a tissue specimen, particularly those coupled to immunoglobulins, can result in error by causing the binding of mobile polymeric carrier molecules according to the invention to the kit even in the absence of ERAC. Repeated washes during the assay would reduce the non-specific binding, but removal of the non-specific agglutinators may be necessary in order to avoid such undesired binding. A simple polystyrene latex surface, for example, can passively delete some of the agglutinators, whereas an Ig G-coated surface provides a better affinity.

Monoclonal antibodies directed against ERAC can provide consistent and reproducible binding. With a proper supply of specific antibody, the present direct binding immunoassay, in contradistinction with competitive binding immunoassay practiced in radioimmunoassay, can be a reliable and very rapid procedure since the incubation time for a kinematic equilibrium needed in competitive binding assays is not presently required.

In accordance with the methods of the present invention, antibody targeting species, either from the usual Ig fraction of the antisera or from monoclonal antibodies, is conjugated respectively with a solid support of a kit as well as optionally with a mobile solid phase, the so called polymeric carrier molecules according to the invention.

The functions of the kit are for the handling and the separation of bound from free antigens, whereas that of the mobile polymeric carrier molecules according to the invention are for the detection of the formed immuno-complexes. Coupling techniques between the antibody protein and various solid phase materials are well developed (see, for example, W. J. Dreyer, U.S. Pat. No. 3,853,987).

In one embodiment of the method of the present invention, the resulting immunocomplex is a multilayered “sandwich” comprising:

Solid support+targeting species, preferably a labelled antibody+ERAC+targeting species, preferably an antibody.

The amount of antibody required for covalent binding, however, can be less than a thousand times that of passive adsorption to a plastic such as polyvinyl chloride and the economics of using such an amount of highly specific targeting species, preferably an antibody, can be prohibitive.

An alternative way of binding that retains some strength of the covalent binding as well as the specificity of targeting species, preferably an antibody, is to bridge the targeting species and the solid phase with a first antibody, an antispecies antibody targeted against the Fc portion of the targeting antibody.

That is, an inexpensive first antibody may initially be covalently bound to the solid phase, and the bound first antibody attracts the species-specific Fc portion of a targeting antibody, leaving the functional epitope of the targeting antibody unaltered with regard to an antigen of ERAC. Bridged with such a first antispecies antibody, the immunoassay of the present invention brings about the following coupling “sandwich” in the case of detection of a species:

Solid support+antispecies antibody+labelled targeting antibody+ERAC+targeting antibody+antispecies antibody.

In the direct binding assay of the present invention, the couplings between the solid support and targeting species, preferably an antibody, as well as optionally the couplings between the polymeric carrier molecules according to the invention and targeting species, preferably an antibody, are prepared in advance, and elements of non-specific agglutination in the sample are removed or deactivated for pre-treatment prior to the direct binding assaying as mentioned above.

Micro Flow System

The kit according to the invention may also be applied in a micro system, such as a micro flow system described in WO 98/10267, one such system being marketed by Torsana Biosensor A/S, Denmark.

The principle behind the technology of a micro flow system is that by controlling the flow rate of at least two guiding streams, a sample stream can be accurately positioned on a target surface.

By controlling the flow ratios between the guiding streams and the sample stream, the sample stream can be focused to a width of a few mm. The sample stream carries the molecules to interact with the surface.

Immobilized lanes of the system are interacted with liquid streams containing unknown samples in the y-dimension.

Thousands of unique intersection points are created where reaction can occur.

The fact that no turbulence occurs in very narrow fluid streams results in diffusion being the only phenomena perturbing the focus of the sample stream. In effect, the technology permits the precise positioning of a liquid stream on a planar surface. In this way it is possible to position material with a precision of a few mm.

The microfluidic system allows for control of very narrow streams of liquid carrying the material (DNA, proteins, cells) to be interacted with the surface of the chip.

The micro flow system enables immobilization of reactant streams, in the present context streams of labelled targeting species and subsequent testing with one or several samples creating a weave with thousands of intersection points where chemical reactions occur and are detected. The entire procedure is performed in a closed fluidic system providing the flexibility in terms of sample—and reactant application, choice of immobilization—and detection chemistries, and array layout.

In particular when using the kit for testing for a plurality of immunological markers, such as providing profiles of immunological markers or a profile of autoantibodies, the invention suitably includes the use of the kit in a microsystem.

In addition to micro systems, the kit according to the invention may also form part of a conventional macro system such as e.g. a lateral flow device.

Lateral Flow Devices

The kit of the present invention may preferably be in the form of a lateral flow device (LFD). A lateral flow device, also known as a lateral flow test or a lateral flow immunochromatographic assay is a simple device intended to detect the presence or absence or quantity of a target analyte, such as a protein or peptide or protein complex or peptide complex or nucleic acid, in sample (matrix). Most commonly lateral flow devices are used for medical diagnostics either for home testing, point of care testing, or laboratory use. Often produced in a dipstick format, lateral flow tests are a form of immunoassay in which the test sample flows along a solid support via capillary action. After the sample is applied to the test it usually encounters a labelled reagent which mixes with the sample and transits the substrate encountering lines or zones which have been pre-treated with a targeting species, such as an antibody. Depending upon the analytes present in the sample, the labelled targeting species can become bound at the test line or zone. Lateral flow tests can operate as either competitive or sandwich assays.

A LFD according to the invention may comprise a solid support enclosed by a casing, such as a hard plastic casing. The casing may preferably have at least an opening or aperture for application of a sample to the solid support (generally termed “sample application aperture”) as well as an opening or aperture or transparent part for allowing observation or reading of the test results and optionally any standards comprised on the solid support (generally termed “test result observation aperture”).

In an embodiment of the invention the solid support of the LFD comprises an application zone for application of the sample, typically through the mentioned sample application aperture of the casing. When applied to the solid support, the sample will flow through the solid support because of capillary forces. In the following, the terms up-stream and down-stream refers to the flow direction of sample in the solid support. Typically, the application zone is situated at one end of the solid support so that the sample substantially only flows in one direction. The solid support further comprises a binding zone down-stream from or overlapping with said application zone. The solid support further comprises a test zone downstream from said binding zone. The solid support may further comprise a control zone down-stream from said test zone.

When the LFD is set up for a sandwich-type assay, the binding zone comprises labelled targeting species capable of binding ERAC. The test zone comprises non-labelled targeting species capable of binding ERAC. In this manner, ERAC in the sample will flow from the application zone to the binding zone. In the binding zone, the ERAC in the sample will be bound by the labelled targeting species, such as a labelled monoclonal anti-ERAC antibody. The ERAC bound by the labelled targeting species will then flow on to the test zone where it will be bound to the non-labelled targeting species. In this way, the labelled targeting species will end up in the test zone (positive result) when the sample comprises ERAC. If no ERAC is present in the sample, no labelled targeting species will end up in the test zone (negative result).

When the LFD is set up for a competitive-type assay, the binding zone will comprise targeting species bound to labelled ERAC. ERAC in the sample will compete for binding to the targeting species, thereby competing with the labelled ERAC. The labelled or non-labelled ERAC bound by the labelled targeting species will then flow on to the test zone where it will be bound to the non-labelled targeting species. The more ERAC contained in the sample, the less label will end up in the test zone. In other words, a sample completely devoid of ERAC will result in a fully labelled test zone, whereas a sample with very much ERAC will result in a test zone with very little or no label.

If the LFD comprises a control zone for demonstrating that the sample has run through the solid support from the application zone through the binding zone and the test zone and beyond, the binding zone will comprise a control protein as well as labelled targeting species capable of binding said control protein. The control zone will then comprise non-labelled targeting species capable of binding the control protein. In this manner, if a sample is applied to the LFD, it will run through the solid support from the application zone and through the binding and test zones to at least the control zone. The labelled targeting species bound to the control protein will be dragged with the sample and end up by being bound by the targeting species in the control zone. In this manner, the control zone will be labelled if the sample has run through the solid support.

The targeting species used for binding to ERAC in the LFD may preferably be an antibody, such as a monoclonal antibody capable of recognising dimers and oligomers of S100A12, including ERAC. However, the antibody does not recognise S100A12 monomers resulting from dissociation of S100A12 dimers and oligomers in the presence of a divalent metal ion chelator such as EDTA, i.e. in the absence of calcium ions. Preferably, the antibody is an antibody specifically binding ERAC. In an embodiment, the antibody is an antibody specifically binding ERAC while at the same time not binding to native S100A12 oligomers.

An embodiment of a kit may preferably comprise:
a zone for applying a body fluid sample possibly comprising ERAC, said zone comprising at least one movable labelled targeting species capable of binding said ERAC, said application zone being in liquid contact with
a zone for testing the presence, amount or concentration of ERAC bound to said targeting species, said zone further comprising a targeting species for immobilizing onto said test zone at least a substantial amount of ERAC comprised in said body fluid sample, and optionally
a positive control zone generating a positive control confirming the transfer of at least part of said body fluid sample from said application zone to said detection zone.

The at least one labelled targeting species comprised in the sample application area, or optionally in a separate binding zone, preferably comprises an antibody comprising at least one label, tag, linker or marker that makes it possible at least to detect the presence of said labelled targeting species, and preferably also makes it possible to quantifiably detect said antibody and/or said labelled targeting species bound to ERAC.

The targeting species of the test zone is preferably also an antibody, but this antibody may not comprise any tag, label or marker. It is thus possible to immobilise onto the test zone an amount of a quantifiably detectable reporter species that accurately reflects the amount of marker present in the body fluid sample. The at least one tag, label or marker used preferably allows both visual detection, by means of the generation of e.g. electromagnetic radiation or a visible colour, and quantification of e.g. the emitted electromagnetic radiation.

Movable targeting species shall be understood to comprise a targeting species capable of moving on, in or through e.g. a solid or semi-solid surface, e.g. when being applied to a lateral flow device comprising a solid support.

In one embodiment of this aspect of the invention there is provided an assay device for detecting ERAC present in a body fluid sample, said device comprising:
a hollow casing having a body fluid sample application aperture and a test result observation aperture,
a bibulous body fluid sample receiving member within said hollow casing to receive said body fluid sample applied to said sample application aperture,
a test strip comprising a dry porous carrier such as nitrocellulose within said casing and extending from said bibulous body fluid sample receiving member to and beyond said test result observation aperture, said dry porous carrier having a test result zone observable through said observation aperture,
at least one of said bibulous body fluid sample receiving member and said test strip containing upstream from said test result zone a detectable targeting species capable of specifically binding ERAC to form a first complex,
said targeting species comprising at least one particulate label, such as a dye sol, a metallic sol or a coloured latex particle, and optionally also at least one fluorescently detectable label, said label being released into a mobile form by said body fluid sample,
   wherein mobility of said label within said test strip is facilitated by either coating at least a portion of said test strip upstream from said test result zone with a material comprising a polysaccharide, or drying said label onto a portion of said test strip upstream from said test zone in the presence of a material comprising a polysaccharide, in an amount effective to reduce interaction between said test strip and said label, and
   wherein said dry porous carrier contains in said test result zone a means for binding said first complex, said means for binding comprising non-labelled targeting species immobilized in said test result zone, and
   wherein migration of said body fluid sample from said bibulous sample receiving member into and through said dry porous carrier conveying by capillarity said first complex to said test result zone of said dry porous carrier whereat said binding means binds said first complex thereby to form a second complex, and
   determining the presence, amount or concentration of said second complex being observable through said test result observation aperture.

In another embodiment there is provided an assay device for detecting ERAC in a body fluid sample, said device comprising a solid support including at least one detectable targeting species on a test area of the solid support, said at least one detectable targeting species being capable of binding ERAC, said targeting species further comprising a liposome or a microcapsule comprising a visible particulate dye compound and optionally also a fluorescently detectable marker.

In yet another embodiment there is provided an assay device comprising
a sample application area comprising a predetermined amount of a targeting species comprising an antibody capable of binding ERAC deposited thereon, said area being in fluid communication with
a reaction zone comprising a mobilizable targeting species comprising an antibody capable of binding said indicator, said targeting species further comprising at least one visually detectable particle and/or at least one fluorescently detectable particle, and
a detection zone comprising a targeting species comprising an antibody capable of binding said indicator,
   wherein, when said body fluid sample comprising ERAC is applied to said sample application area, a threshold amount of the indicator is bound to said antibody and thereby prevented from binding to the antibody being present in the reaction zone, and
   wherein the ERAC remaining unbound in said body fluid sample passes from the sample application area through said reaction zone, where it is bound to said mobilizable targeting species comprising i) an antibody capable of binding said indicator, and ii) at least one visually detectable particle and/or at least one fluorescently detectable particle, and
   wherein ERAC bound to the mobilizable targeting species is brought into contact with the detection zone, where ERAC is bound to said targeting species comprising said antibody capable of binding ERAC, and
   wherein said binding of ERAC results in immobilization of said mobilizable targeting species further comprising i) an antibody capable of binding ERAC, and ii) at least one visually detectable particle and/or at least one fluorescently detectable particle.

The present invention employs targeting species, labelling species, and more generally molecular species. The term “molecular species” in the context of the present invention is used to denote, for example: molecules or ionic species which serve as labels or markers (such as enzymes, or fluorescent or luminescent species); or molecules which serve as targeting species, i.e. molecules which are capable of binding selectively or specifically to one or more target molecules, moieties, receptors or epitopes (examples of such targeting species being haptens or hapten conjugates, antigens, antibodies, nucleotide sequences and hormones). The invention in one particular embodiment relates to simultaneously or sequentially using any one or both of a first targeting species and a second targeting species including polyclonal and monoclonal antibodies that may be, respectively, i) identical or non-identical, and ii) specific for the same or different epitopes of antigenic determinants characteristic for ERAC.

Molecular species according to the invention are to be found among numerous different types of substances, examples being: proteins, such as ferritin, phycoerythrins, phycocyanins or phycobilins; enzymes, such as horseradish peroxidase, alkaline phosphatase, glucose oxidases, galactosidases or ureases; toxins; drugs; dyes; fluorescent, luminescent, phosphorescent or other light-emitting substances; metal-chelating substances, such as iminodiacetic acid, ethylenediaminetetraacetic acid (EDTA), diethylenetriaminepentaacetic acid (DTPA) or desferrioxamine B; substances labelled with a radioactive isotope; or substances labelled with a heavy atom.

Many molecular species will be able to serve as labelling species in conjugates according to the invention. Additional examples of labelling species are listed herein immediately below.

i) Fluorescent substances selected from, e.g., fluorescein (suitably as fluorescein isothiocyanate, FITC), fluoresceinamine, 1-naphthol, 2-naphthol, eosin, erythrosin, morin, o-phenylenediamine, rhodamine and 8-anilino-1-naphthalenesulfonic acid.
ii) Radioactive isotopes of relevance may be selected, for example, among isotopes of hydrogen (i.e. tritium, 3H), carbon (such as 14C), phosphorus (such as 32P), sulfur (such as 35S), iodine (such as 131I), bismuth (such as 212Bi), yttrium (such as 90Y), technetium (such as 99Tc), palladium (such as 109Pd) and samarium (such as 153Sm).
iii) Heavy atoms of relevance may be selected, for example, among Mn, Fe, Co, Ni, Cu, Zn, Ga, In, Ag, Au, Hg, I, Bi, Y, La, Ce, Eu and Gd. Gold (Au) may be used in combination with silver (Ag) as an enhancement reagent and Au is a particularly useful heavy atom in many cases.

Molecular species may also be in the form of targeting species capable of selectively binding to, or selectively reacting with, a complementary molecule or a complementary structural region of a material of biological origin. Examples of relevant targeting species are, for example: antigens; haptens; monoclonal or polyclonal antibodies; gene probes; natural or synthetic oligo- or polynucleotides; certain natural or synthetic mono-, oligo- or polysaccharides; lectins; avidin or streptavidin; biotin; growth factors; hormones; receptor molecules; or protein A or protein G. For examples of appropriate antibodies, reference is made to the working examples given herein. Examples of relevant hormones may be selected from steroid hormones (e.g. estrogen, progesterone or cortisone), amino acid hormones (e.g. thyroxine) and peptide and protein hormones (e.g. vasopressin, bombesin, gastrin or insulin).

The present invention may in one embodiment employ standard immunohistochemical or cytochemical detection procedures for the detection of ERAC, or any suitable modifications of such procedures. Accordingly, the invention may employ any assay resulting in the recognition of an antigenic determinant mediated by an immunochemical reaction of the antigenic determinant with a specific so-called primary antibody capable of reacting exclusively with the target antigenic determinant in the form of ERAC.

The primary antibody is preferably labelled with an appropriate label capable of generating—directly or indirectly—a detectable signal. The label is preferably an enzyme, an isotope, a fluorescent group or a heavy metal such as gold.

In another embodiment, the invention employs the detection of the primary antibody by immunochemical reaction with specific so-called secondary antibodies capable of reacting with the primary antibodies. In this case the secondary antibodies are preferably labelled with an appropriate label such as an enzyme, an isotope, a fluorescent group or a heavy metal such as gold.

In yet another embodiment, the present invention employs a so-called linker antibody as a means of detection of ERAC. This embodiment exploits that the immunochemical reaction between the target antigenic determinant in the form of ERAC and the primary antibody is mediated by another immunochemical reaction involving the specific linker antibody capable of reacting simultaneously with both the primary antibody as well as another antibody to which enzymes have been attached via an immunochemical reaction, or via covalent coupling and the like.

In yet another embodiment according to the present invention, the immunochemical reaction between a target antigenic determinant in the form of a ERAC and the primary antibody, or alternatively, between the primary antibody and the secondary antibody, is detected by means of a binding of pairs of complementary molecules other than antigens and antibodies. A complementary pair such as e.g. biotin and streptavidin is preferred. In this embodiment, one member of the complementary pair is attached to the primary or secondary antibody, and the other member of the complementary pair is contacted by any suitable label such as e.g. an enzyme, an isotope, a fluorescent group or a heavy metal such as gold.

When an enzyme label is used as a labelling species, the ERAC bound to a solid support as described herein above is treated with a substrate, preferably a colour developing reagent. The enzyme reacts with the substrate, and this in turn leads to the formation of a coloured, insoluble deposit at and around the location of the enzyme. The formation of a colour reaction is a positive indication of the presence of the ERAC in the sample.

When a heavy metal label such as gold is used, the sample is preferably treated with a so-called enhancer in the form of a reagent containing e.g. silver or a similar contrasting indicator. Silver metal is preferably precipitated as a black deposit at and around the location of the gold. When a fluorescent label is used, a developing reagent is normally not needed.

It may be desirable to introduce at least one washing step after which some of the constituents of the sample are preferably coloured by reaction with a suitable dye resulting in a desirable contrast to the colour provided by the labeling species in question. After an optional final washing step, the specimen is preferably coated with a transparent reagent to ensure a permanent record for the examination.

Detection of the labelling species in question preferably indicates both the localization and the amount of the target antigenic determinant in the form of ERAC. The detection may be performed by visual inspection, by light microscopic examination in the case of enzyme labels, by light or electron microscopic examination in the case of heavy metal labels, by fluorescence microscopic examination, using irradiated light of a suitable wavelength in the case of fluorescent labels, and by autoradiography in the case of an isotope label. Detection of the presence of the ERAC—and preferably also the amount of the indicator—by visual inspection of the sample is preferred.

In a particularly preferred embodiment, the visual detection is based on a cut-off point above which one colour indicates the presence of the ERAC above a certain minimum amount (cut-off point), and below which cut-off point another colour indicates that the ERAC is present in an amount of less than that indicated by the cut-off point. When fluorescent markers are used the amounts of ERAC detected can be directly correlated with the fluorescence measured by a detection unit.

The preferred statistical quality parameters for the present invention when the test is capable of detecting an amount of ERAC as specified above within a predetermined time period may be summarised as follows:

Sensitivity: at least 80%, such as at least 85%, such as at least 90%
Specificity: at least 80%, such as at least 85%, such as at least 90%
Positive predictive value: at least 80%, such as at least 85%, such as at least 90%
Negative predictive value: at least 80%, such as at least 85%, such as at least 90%, more preferably at least 99.5%

The positive and the negative predictive values are closely related to the prevalence of the clinical condition in the population, to be tested. In the present context it is preferred that the statistical calculations are based on a type of population which is realistic for using the test. Thus, the statistical calculations are not based only on a population known to have acquired the clinical condition, but also on individuals that might turn out as negatives for the clinical condition. Due to the validity and sensitivity of the kit according to the present invention the kit is particular suitable for testing of populations having a prevalence of the condition being tested for less than 100%, such as less than 90%, such as less than 80%, more preferably less than 70%, even more preferred less than 60%, such as about 50%.

### EXAMPLES

### Example 1

### Chromatographic and Enzyme Immunoassays for Assessment of ERAC

A serum sample is run on a gel permeation chromatography, for instance a 1.6×60 cm column of the type High-Load Superdex-75 from Pharmacia, Sweden, using a buffer consisting of 50 mM tris, 150 mM sodium chloride and 5 mM ETTA, pH 8.0. A flow rate of 1 ml/min is chosen. Proteins with high molecular weights are eluted earlier than those with low molecular weights. ERAC and A12 in fractions eluted from the column are assessed by the ELISA described below. Typically, A12 with a normal molecular configuration of 10 kDa will elute when about 68 ml buffer has passed through the column, while ERAC will elute when only about 55 ml buffer has passed through. The difference in MW of normal A12 and ERAC is so great that there will be no overlap between the factions. A common and consistent way of expressing elution volumes is to express the elution volume as a fraction of one homogenous protein in the sample, typically albumin. Using the column mentioned above, the ratio between the elution volumes of ERAC and albumin is about 0.82. In contrast, the ratio for normal A12 is about 1.32.

Alternatively, ERAC can be distinguished from normal A12 by use of anion exchange chromatography as shown in FIG. 5. A serum sample is applied on a column packed for instance with a DEAE-Sepharose FastFlow gel from Pharmacia, Sweden, equilibrated with 25 mM sodium barbital buffer pH 8.8. Normal A12 is eluted from the column by addition of 75 to 100 mM sodium chloride, while ERAC is eluted when the sodium chloride concentration is increased to 200 mM. In stead of an anion exchange column, the anion exchanger can be a membrane, for instance as a bottom of a microwell so that after ERAC has been bound, its presence can be shown for instance by use of antibodies labelled with enzymes, fluorochromes or coloured particles. Other types of chromatography can be used, for instance cation exchange or hydrophobic interaction chromatography or chromatofocusing by choosing buffers and media allowing the separation of ERAC from normal A12.

The following enzyme immunoassay (ELISA) can be used for assessment of ERAC: Wells in a microtiterplate, for instance with 96 wells taking 300 microliters solution, are incubated with 150 microliters of an IgG fraction of rabbit anti-ERAC diluted, for instance 1:500, in phosphate buffered saline pH 7.4 for at least 18 hours. Alternatively monoclonal antibodies against ERAC can be used in a suitable concentration, for instance 10 micrograms per ml. The optimal dilution will depend on the titer of the anti-ERAC in the rabbit antiserum and of the affinity between ERAC and the antibodies; the latter also applies for monoclonal antibodies. During the incubation anti-ERAC antibodies will bind to the walls of the wells. To prevent evaporation of the water the wells must be covered by use of adhesive tape. Before use, the wells must be washed four times, each time with 250 microliters per well, with PBS containing 0.5 ml Tween-20 per liter.

50 microliters standards, consisting of purified ERAC at concentrations between 400 and 6.25 ng/ml in a buffer containing 50 mM tris, 150 mM sodium chloride, 0.5 mM magnesium chloride, 2.5 mM potassium chloride, 5 mM calcium chloride, 10 g/l bovine serum albumin, 0.5 ml Tween-20 per liter, pH 8.0, are applied in designated wells. Serum samples, 40 microliters mixed with 10 microliters 100 mM solution of potassium ETTA are added to separate wells. Both standards and samples are tested in duplicate. The microplate is covered and shaken, about 1000 rpm, at room temperature for about 60 minutes. Subsequently, the wells are washed as described above. To each well is then added 50 microliters of alkaline phosphatase (ALP) conjugated, affinity purified ant-ERAC in a suitable dilution, for instance 1:500; the plate is incubated with shaking for 60 minutes again. After four times washing as above, 100 microliters of a suitable enzyme substrate, for instance para-nitrophenylphosphate, 0.1 mg/ml, dissolved in a buffer with 10% diethanolamine, 0.1 g/1 magnesium chloride, 0.1 g/l thimerosal, pH 9.5. The enzymatic reaction generates a yellow colour intensity of which reflects the concentration of ERAC. A reading is performed by use of a spectrophotometer designed for microplates. The ERAC concentrations are calculated by a computer connected to or built into the reader or from a manual standard curve where the optical densities (O.D., reflecting the colour intensities) of the standards are plotted against their known concentrations. A typical standard curve is shown in FIG. 6.

A serum sample containing ERAC will give an O.D. corresponding to an ERAC concentration higher than that of the 6.25 ng/ml standard, while samples without ERAC will give lower optical densities.

Alternatively, the ERAC concentration can be determined by a rapid test, for instance by use of an immunochromatographic method based upon the lateral flow principle, often abbreviated as an LFT test. An ERAC LFT test consists of the following: specific anti-ERAC antibodies obtained by immunizing experimental animals with purified ERAC are applied as a narrow stripe across the middle of a membrane consisting of nitrocellulose or similar protein binding membrane. The antibodies will bind irreversible to the membrane which will typically have a dimensions of 5×60 mm. Residual protein binding sites on the membrane is blocked by incubation with an unrelated, animal protein, for instance bovine albumin. To one end of the membrane is attached a second membrane, the sample application pad, dimension about 5×5 mm in which colloid gold particle labelled anti-ERAC has been applied and dried. Alternatively, the antibodies can be labelled with other types of coloured particles, for instance made of latex. This membrane can consist of filter paper, glass fibres or similar suitable material. To the other end of the long membrane is attached a 5×5 mm pad of water absorbing filter paper When an ERAC containing solution, for instance 100 microliters of serum or chromatographic fractions thereof, are applied on the sample application pad membrane, the labelled antibodies will be solubilised and react with ERAC in the sample; the antigen/antibody complexes will then diffuse into the long membrane towards the water absorbing pad, and bind to the stripe of antibodies across the middle of the membrane; in this way a coloured line will appear across the membrane. The staining intensity of the line will, within a certain range, be proportional to the concentration of ERAC, and can be measured by a photometer. In FIG. 7 is shown an example of an LFT test where one sample with high (500) and one with low (31.3) concentration were tested. By use of such aN LFT test the presence of ERAC in high molecular fraction from gel permeation chromatography can be demonstrated.

### Example 2

### Direct Assessment of ERAC by Use Immunoassays

Many rabbits immunized with purified ERAC produce antibodies against a single antibody binding site or epitope on the protein. Some monoclonal antibodies may also bind to a single epitope. Many commonly used immunoassays like ELISA, LFT, agglutination, nephelometry or immunoprecipitation require that at least two epitopes are present on the antigen, for instance a protein. When anti-ERAC antibodies reacting with a single epitope are used a negative reaction will be found, i.e. no immune complexes are generated, unless S100A12 is present as dimers, oligomers, polymers or heterocomplexes containing at least two A12 monomers. In normal human serum A12 is present mostly as calcium dependent oligomers like dimers or hexamers, see FIG. 1, and they give a strong signal for instance when an ELISA is used. The reaction becomes negative if a calcium chelating substance like EDTA is added to the sample and the assay buffer. The EDTA binds calcium so that oligomers dissociate into monomers as shown in FIG. 2. In fact, the presence of the monomeric fraction can only be shown by the ELISA if calcium is added to the assay buffer, for instance to final concentration of 5 mM, so that oligomers can be formed again. In serum samples containing ERAC a positive reaction will be found even in the presence of EDTA, for instance at a concentration of 5 mM. This is the very reason behind the name ERAC: EDTA Resistant A12 Complex.

A test showing the presence of ERAC can therefore by the use of many different immunoassays, for instance ELISA, immunofluorescence, chemoluminescence, immunoflowcytometry, LFT, agglutination of antibody coated cells or particles, nephelometry or immunoprecipitation in the presence of EDTA. The use of EDTA at a suitable concentration, for instance 5 mM, will prevent a reaction by monomeric A12 while ERAC present in serum from certain patients will give a positive reaction.

### Example 3

### Detection of ERAC in Serum

Selected serum samples from a number of different groups of patients and healthy controls have been examined by Gel Permeation Chromatography (GPC) for the presence of ERAC.

Rheumatoid Arthritis

In a cohort of rheumatoid arthritis (RA) patients (n=129) 17 patients (13.2%) had cardiovascular (CV) disease. The presence of CV disease was based on the patient's history and clinical evaluation as well as by review of the record for relevant confirmative procedures. Hence CV disease was considered present if the patient had been diagnosed with ischaemic heart disease (i.e. angina pectoris or myocardial infarction) by a cardiologist, congestive heart failure confirmed by echocardiography, stroke confirmed by computertomography or intermittent claudications of lower limbs confirmed with angiogram.

We have examined 24 serum samples from this cohort, selected on the basis of high serum concentration of S100A12. Also a few samples with low (normal) S100A12 concentrations were examined. We found ERAC in 21 of the 24 samples.

Examined by GPC: 24 patients

Of the 21 with ERAC present:

ERAC present and CVD present: 10 patients

ERAC present and no CVD: 11 patients

Of the 3 without ERAC:

No ERAC but CVD present: 1 patient

No ERAC and no CVD: 2 patients

Healthy Blood Donors

In a cohort of 150 blood donors (all presumed healthy):

Examined by GPC: 9 persons

ERAC present: 2 persons*

No ERAC: 7 persons

*) Both had serum concentrations of S100A12 in the range of 6 times above the suggested upper reference interval.

Healthy Persons Exercising:

Two healthy persons running 3 km. Temporarily very high concentrations of S100A12 shortly after the exercise, but no ERAC present in these samples.

Normal and preeclamptic pregnancies and pregnant women with glomerulonephritis: In a cohort of women with normal pregnancy and preeclamptic pregnant women:

Normal pregnancies: n=23

Examined by GPC: 3 pregnant women

No ERAC: 3 pregnant women

Preeclamptic women: n=23

Examined by GPC: 4 patients

ERAC present: 1 patient

No ERAC: 3 patients

Pregnant woman with glomerulonephritis*: n=1

No ERAC: 1 patient

*) Also high concentration of S100A12 (in the range of 6 times above the suggested upper reference interval).

Primary Sjöegren's Syndrome:

From a cohort of 142 patients:

Examined by GPC: 6 patients

ERAC present: 5 patients

No ERAC: 1 patient

### Example 4

### Lateral Flow Device

An embodiment of the present invention is a lateral flow immunochromatographic test (LFT test) comprising a membrane strip made of a suitable protein binding material, for instance nitrocellulose (NC), for instance 0.1 mm thick, 60 mm long and 6 mm wide. Antibodies against S100A12 are irreversibly bound as a line, for instance 0.5 mm thick, by pipetting the antibody solution in a suitable concentration, for instance 2 microg/ml, across the central part of the strip; this will be called the “Test zone”. At some distance, for instance 10 mm away from the Test zone, a similar zone of antibodies against an irrelevant protein, for instance goat gammaglobulin, is bound; this zone will be called the “Control zone”. Any remaining protein binding sites on the strip is blocked by incubation with another suitable protein, for instance casein, for a suitable period of time at a suitable temperature, for instance one hour at room temperature. In a position close to one end of the strip, called the sample application region, another suitable membrane, for instance made of glass fibre, of a suitable size, for instance 1 mm thick, 10 mm long and 6 mm wide, is attached; this membrane can suitably be called the “sample application pad”. Before use this pad shall be soaked with antibodies suitably labelled with coloured particles, for instance colloid, in a suitable concentration, for instance 1 micrograms per millilitre; the antibodies must be a mixture of anti-S100A12 and antibodies against the protein used in the Control zone as well as a suitable concentration of the protein that will bind to the antibodies in the Control zone. After soaking for a suitable period of time at a suitable temperature, for instance one hour at room temperature, the sample application pad is allowed to dry, for instance in front of a fan at room temperature for one hour. At the other end of the strip, i.e. opposite to the sample application pad, is attached a pad of water absorbent material, for instance 2 mm thick, 6 mm wide and 10 mm long, for instance made of filter paper. To avoid the membrane absorbing water from the air, the strip with attachment should be packed in a water vapour tight pouch also containing a desiccant, for instance dried silica. For easy handling, the strip may be put in a cassette, for instance made of plastic, with openings for sample application and inspection of the Test and Control zones. When a sample containing S100A12 is added, for instance serum or dilutions hereof, for instance a volume of 100 microliters, the proteins in the sample pad will be dissolved and diffuse into the NC strip towards the absorbent pad. The labelled antibodies against S100A12 will bind to any S100A12 molecules or complexes containing this protein; when such antibody-antigen complexes reach the Test zone they will be bound by the antibodies there and give it a colour like that of the label on the antibodies. The colour intensity will increase will increase with time and reach a maximum after about one hour. For quantitative assay, an incubation period of about 10 minutes may be preferred to have a quick result as expected from a rapid test. In addition to the Test zone, a similarly coloured Control zone will appear in the test strip in a position corresponding to the zone where Control antibody had been applied. This zone serves the function of confirming that a sample has been applied, that the dissolution of dried protein occurred and that the diffusion into the NC membrane took place. For quantitative purposes it is possible to use the ratio of staining intensities of Test zone and Control zone to compensate for possible variability in the sample pad or NC membrane.

### Example 5

### Use of a Lateral Flow Test for Detection of ERAC

A qualitative test: Such a test is intended to show whether or not a sample contains ERAC. The test is based upon the use of monoclonal antibodies reacting with a single, unique epitope on the S100A12 molecule. Serum normally contains calcium dependent oligomers of S100A12 so that at least two epitopes are available for antibody binding, including those labelled with coloured particles. Sera from nearly all individuals will therefore give a positive reaction, i.e. a coloured Test line in the LFT. In contrast, if samples are added Ethylene Diamine Tetra acetic Acid (EDTA) to a suitable concentration, for instance 10 mM, only ERAC positive samples will give a positive test reaction. A positive ERAC test will require that a distinct Test line appears at the time of reading, for instance after 10 minutes even after addition of EDTA. A more objective reading can be performed by use of a reader instrument. FIG. 6 shows the test patterns when an ERAC positive and an ERAC negative serum sample were tested. The arrows show the test lines in the ERAC positive and ERAC negative reaction.

### Example 6

### A Quantitative Test

The staining intensity of the Test zone on the ERAC lateral flow test described above can be determined by photometric instruments. FIG. 7 shows a typical scanning curve when a LFT test strip has been scanned. As shown in FIG. 7, the Control zone gives peaks of equal size irrespective of the protein concentrations (0 to 500 ng/ml), while the Test zone size increases with increasing concentration of S100A12 in the sample.

A scanning can be performed by use of different types of scanners, including standard office document scanners, a standard computer, for instance a Lap-Top, and a computer program for image analysis as shown above. Alternatively, a picture of the LFT test with a Test zone can be taken by the camera of a mobile telephone and sent as an MMS file to a central server computer for image analysis; the quantitative result can be sent back to the mobile telephone within a short time, typically 20 seconds.

A typical standard curve obtained when samples with S100A12 concentrations between 50 and 2000 ng/ml were tested and read by use of a scanner is shown in FIG. 8. The presence of ERAC can be defined by a Test zone corresponding to a concentration above certain level to be determined according to a defined regimen of sampling, sample handling and the LFT procedure. For the latter, the type and concentration of antibodies are of special importance. Alternatively, Test zone staining intensity can be determined by comparison with a series of lines on a printed visual analogue scale. The human eye can see differences between staining intensities if they are greater than about 15%. FIG. 9 shows the correlation between scanner readings and those from visual analogue readings of the same LFT test strips.

1-150. (canceled)

151. A polypeptide complex comprising a plurality of S100A12 monomers or oligomers, wherein a monomer of S100A12 has the amino acid sequence TKLEEHLEGI VNIFHQYSVR KGHFDTLSKG ELKQLLTKEL ANTIKNIKDK AVIDEIFQGL DANQDEQVDF QEFISLVAIA LKAAHYHTHK E (SEQ ID NO:1), or is substantially identical to SEQ ID NO:1, and wherein the S100A12 monomers or oligomers of said complex do not dissociate in the presence of a low concentration of EDTA.

152. A host cell comprising the polypeptide complex of claim 151.

153. A composition comprising the polypeptide complex of claim 151.

154. A method for detecting the polypeptide complex of claim 151 in a sample, comprising the steps of:
i) providing a sample,
ii) removing divalent metal ions from the sample, thereby reducing or eliminating the amount of free divalent metal ion in the sample,
iii) providing a labeled first targeting species capable of directly detecting said polypeptide complex when present in the sample,
iv) bringing the sample into contact with said labeled first targeting species, and
v) detecting the presence of said polypeptide complex bound to said labeled first targeting species.

155. The method of claim 154 further comprising the step of:
i) quantifying the amount of said polypeptide complex bound to said labeled first targeting species.

156. A method for quantifying the amount of the polypeptide complex of claim 151 present in a sample, comprising the steps of:
i) providing a kit,
ii) providing a sample,
iii) bringing the sample into contact with the kit,
iv) detecting the presence of said polypeptide complex in the sample, and
v) quantifying the amount of said polypeptide complex detected in the sample.

157. A method for profiling a sample, comprising the steps of:
i) detecting the presence of the polypeptide complex of claim 151 in the sample using the method of claim 154 or quantifying the amount of the polypeptide complex of claim 151 in the sample using the method of claim 156, and
ii) qualitatively or quantitatively detecting the presence of at least one other immunological marker in the sample.

158. A method for monitoring the presence of the polypeptide complex of claim 151 in a sample, comprising the steps of:
i) detecting the presence of said polypeptide complex in the sample using the method of claim 154, or quantifying the amount of said polypeptide complex in the sample using the method of claim 156, and
ii) repeating step (i), optionally at predetermined intervals.

159. A method for diagnosing a clinical condition, comprising the steps of:
i) detecting the presence of the polypeptide complex of claim 151 in a sample using the method of claim 154, quantifying the amount of the polypeptide complex of claim 151 in a sample using the method of claim 156, or profiling a sample using the method of claim 157, or monitoring the presence of the polypeptide complex of claim 151 in a sample using the method of claim 158, and
ii) diagnosing said clinical condition.

160. A method for prognosing the outcome or progress or development or relapse or remittance of a clinical condition in an individual, comprising the steps of:
i) detecting the presence of the polypeptide complex of claim 151 in a sample using the method of claim 154, quantifying the amount of the polypeptide complex of claim 151 in a sample using the method of claim 156, or profiling a sample using the method of claim 157, or monitoring the presence of the polypeptide complex of claim 151 in a sample using the method of claim 158, and
ii) determining the prognosis of said individual.

161. A method for identifying a compound that competes with the polypeptide complex of claim 151 for binding to a receptor, comprising the steps of:
i) providing said polypeptide complex,
ii) providing a receptor to which said polypeptide complex binds,
iii) providing at least one other compound, and
iv) testing the binding of said at least one other compound to the receptor in the presence of said polypeptide complex.

162. A method for diagnosing a cardiovascular (CV) disease in a patient, comprising the steps of:
i) obtaining a body fluid sample from a patient,
ii) incubating said sample with a divalent metal ion chelator, and
iii) detecting the polypeptide complex of claim 151 in said sample, wherein the detection of said polypeptide complex correlates positively with the presence of a CV disease in said patient.

163. A method for diagnosing rheumatoid arthritis in a patient, comprising the steps of:
i) obtaining a body fluid sample from a patient,
ii) incubating said sample with a divalent metal ion chelator, and
iii) detecting the polypeptide complex of claim 151 in said sample, wherein the detection of said polypeptide complex correlates positively with the presence of rheumatoid arthritis in said patient.

164. A method for removing the polypeptide complex of claim 151 from a body fluid of a patient in need thereof, comprising the steps of:
i) detecting said polypeptide complex in said body fluid, and
ii) removing said polypeptide complex from said body fluid.

165. A method for identifying an antagonist of the binding of the polypeptide complex of claim 151 to RAGE (Receptor for Advanced Glycosylation Endproducts), comprising the steps of:
i) contacting a candidate antagonist with said polypeptide complex, and
ii) determining the binding affinity of said candidate antagonist,
wherein a high binding affinity of said candidate antagonist to said to polypeptide complex indicates that said candidate antagonist is capable of binding to said polypeptide complex and competitively inhibit the binding of said polypeptide complex to RAGE.

166. A method for treatment of a clinical condition, comprising the step of:
i) reducing in an individual in need thereof the amount of the polypeptide complex of claim 151 that is present in a body fluid of said individual.

167. A method for treatment of a clinical condition, comprising the step of:
i) administering to an individual in need thereof a compound capable of competing with the polypeptide complex of claim 151 for binding to a receptor.

168. A method according to any of claim 166 or 167, wherein said method further comprises treating said clinical condition using an anti-inflammatory agent.

169. A method according to any one of the claim 166 or 167, wherein said clinical condition is selected from the group consisting of chronic and acute inflammatory conditions, auto-immune diseases, cancer, kidney diseases or malfunctions, cardiovascular disease, and infection.

170. A method according to any one of the claim 166 or 167, wherein said clinical condition is rheumatoid arthritis.
